(19) United States
(12) Patent Application Publication
(10) Pub. No.: US 2005/0238686 A1
(43) Pub. Date: Oct. 27, 2005

(54) Coating for implantable devices and a method of forming the same

(75) Inventors: Syed F.A. Hossainy, Fremont, CA (US); Steven Z. Wu, Hillsborough, NJ (US); Manish Gada, Santa Clara, CA (US); Anthony Andreacchi, San Jose, CA (US)

Correspondence Address: SQUIRE, SANDERS & DEMPSEY LLP, 1 MARITIME PLAZA, SUITE 300, SAN FRANCISCO, CA, 94111, US

(73) Assignee: Advanced Cardiovascular Systems, Inc.

(21) Appl. No.: 11/089,763

(22) Filed: Mar. 25, 2005

Related U.S. Application Data
(63) Continuation-in-part of application No. 10/320,935, filed on Dec. 16, 2002, now Pat. No. 6,908,624.
Application No. 10/320,935 is a continuation-in-part of application No. 09/750,595, filed on Dec. 28, 2000, now Pat. No. 6,790,228.
Application No. 09/750,595 is a continuation-in-part of application No. 09/470,559, filed on Dec. 23, 1999, now Pat. No. 6,713,119.

(51) Int. Cl. B05D 3/00; A61L 2/00; A61F 2/00

(52) U.S. Cl. 424/423; 427/2.1

(57) ABSTRACT

Coatings for implantable devices or endoluminal prosthesis, such as stents, are provided, including a method of forming the coatings. The coatings can be used for the delivery of an active ingredient or a combination of active ingredients.

### CROSS-REFERENCE

This is a continuation-in-part of U.S. patent application Ser. No. 10/320,935, filed on Dec. 16, 2002, allowed, which is a continuation-in-part of U.S. patent application Ser. No. 09/750,595, filed on Dec. 28, 2000, which is a continuation-in-part of U.S. patent application Ser. No. 09/470,559 filed on Dec. 23, 1999.

### BACKGROUND

1. Field of the Invention

The invention relates to coatings and methods of forming the coatings on implantable devices or endoluminal prostheses, such as stents.

2. Description of the Background

Percutaneous transluminal coronary angioplasty (PTCA) is a procedure for treating heart disease. A catheter assembly having a balloon portion is introduced percutaneously into the cardiovascular system of a patient via the brachial or femoral artery. The catheter assembly is advanced through the coronary vasculature until the balloon portion is positioned across the occlusive lesion. Once in position across the lesion, the balloon is inflated to a predetermined size to radially press against the atherosclerotic plaque of the lesion for remodeling of the vessel wall. The balloon is then deflated to a smaller profile to allow the catheter to be withdrawn from the patient's vasculature.

A problem associated with the above procedure includes formation of intimal flaps or torn arterial linings which can collapse and occlude the conduit after the balloon is deflated. Vasospasms and recoil of the vessel wall also threaten vessel closure. Moreover, thrombosis and restenosis of the artery may develop over several months after the procedure, which may require another angioplasty procedure or a surgical by-pass operation. To reduce the partial or total occlusion of the artery by the collapse of arterial lining, and to reduce the chance of the development of thrombosis and restenosis, an expandable, intraluminal prosthesis, one example of which includes a stent, is implanted in the lumen to maintain the vascular patency.

Stents are used not only as a mechanical intervention but also as a vehicle for providing biological therapy. As a mechanical intervention, stents act as scaffoldings, functioning to physically hold open and, if desired, to expand the wall of the passageway. Typically stents are capable of being compressed, so that they can be inserted through small cavities via catheters, and then expanded to a larger diameter once they are at the desired location. Mechanical intervention via stents has reduced the rate of restenosis as compared to balloon angioplasty; but restenosis is still a significant clinical problem with rates ranging from 20-40%. When restenosis does occur in the stented segment, its treatment can be challenging, as clinical options are more limited as compared to lesions that were treated solely with a balloon.

Biological therapy can be achieved by medicating the stents. Medicated stents provide for the local administration of a therapeutic substance at the diseased site. In order to provide an efficacious concentration to the treated site, systemic administration of such medication often produces adverse or toxic side effects for the patient. Local delivery is a preferred method of treatment in that smaller total levels of medication are administered in comparison to systemic dosages, but are concentrated at a specific site. Local delivery thus produces fewer side effects and achieves more favorable results. The embodiments of the present invention provide stent coatings for local delivery of drugs.

### SUMMARY

In accordance with one aspect of the present invention, a method of forming a coating for an implantable device is provided. The method includes forming a primer layer comprising a polymer on at least a portion of a surface of an implantable device, wherein the primer layer has a weight measurement of X, and forming a reservoir layer comprising a polymer and an active ingredient on at least a selected portion of the primer layer, wherein the reservoir layer has a weight measurement of Y and wherein X/Y is equal to or greater than 0.25. In one embodiment, the coating has a drug loading equal to or greater than 30%. In another embodiment, the method further comprises forming asperities on the surface of the primer layer preceding the formation of the reservoir layer. In yet another embodiment, the primer layer includes at least a region having a degree of porosity.

In another aspect, a method of forming a coating for an implantable device is provided. The method includes forming a primer layer comprising a polymer on at least a portion of a surface of an implantable device, wherein the primer layer has a thickness X and forming a reservoir layer comprising a polymer and an active ingredient on at least a selected portion of the primer layer, wherein the reservoir layer has a thickness Y and wherein X/Y is equal to or greater than 0.25. In one embodiment, the thickness X is about 0.5 microns to about 3 microns and the thickness Y is about 1 micron to about 10 microns.

In yet another aspect of the present invention, an implantable device is provided comprising a coating for delivery of an active ingredient. The coating includes a primer region comprising a polymer on at least a portion of a surface of an implantable device, wherein the primer region has a thickness X, and a reservoir region comprising a polymer and an active ingredient on at least a selected portion of the primer region, wherein the reservoir region has a thickness Y, and wherein X/Y is equal to or greater than 0.25. The thickness X is measured from the outer surface of the primer region to the surface of the implantable device prior to the migration of the active ingredient from the reservoir region to the primer region. In one embodiment, the coating further includes a barrier region located on at least a selected portion of the reservoir region for reducing the rate at which the active ingredient is released from the coating after insertion of the device into a body of a patient. In another embodiment, the primer region includes a porous matrix extending from the interface of the primer region and the reservoir region into the primer. In some invention embodiments, X/Y, with X/Y representing the thickness or the mass of the respective layers, can be 0.01 to 0.2; 0.05 to 0.2; 0.01 to 20; 0.05 to 20; 0.01 to 1; 0.05 to 1; or 0.01 to 0.05.

In another aspect, a stent comprising a coating for delivery of an active ingredient is provided, wherein the coating includes a primer region comprising a polymer and a reservoir region comprising a polymer and an active ingredient, and wherein the thickness or the weight of the primer region is sufficiently high so as to allow drug loading of 30% in the reservoir region without causing the coating to significantly crack when the stent is expanded.

### BRIEF DESCRIPTION OF THE FIGURES

FIGS. 1A-1E illustrate coatings in accordance with some of the embodiments of the present invention;

FIGS. 2A and 2B illustrate coatings having different layers;

### DETAILED DESCRIPTION OF THE EMBODIMENTS

This document fully incorporates by reference the entire text of U.S. patent application Ser. No. 10/320,935, filed on Dec. 16, 2002, allowed, U.S. patent application Ser. No. 09/750,595, filed on Dec. 28, 2000, and U.S. patent application Ser. No. 09/470,559 filed on Dec. 23, 1999.

For ease of discussion, the methods and apparatus detailed herein will be described with reference to a coating for a stent. However, the implantable device coated in accordance with embodiments of the present invention may be any suitable medical substrate that can be implanted in a human or veterinary patient. Examples of such implantable devices include self-expandable stents, balloon-expandable stents, stent-grafts, grafts (e.g., aortic grafts), artificial heart valves, cerebrospinal fluid shunts, pacemaker electrodes, and endocardial leads (e.g., FINELINE and ENDOTAK, available from Guidant Corporation). The underlying structure of the device can be of virtually any design. The device can be made of a metallic material or an alloy such as, but not limited to, cobalt chromium alloy (ELGILOY), stainless steel (316L), “MP35N,” “MP20N,” elastinite (Nitinol), tantalum, nickel-titanium alloy, platinum-iridium alloy, gold, magnesium, or combinations thereof. “MP35N” and “MP20N” are trade names for alloys of cobalt, nickel, chromium and molybdenum available from standard Press Steel Co., Jenkintown, Pa. “MP35N” consists of 35% cobalt, 35% nickel, 20% chromium, and 10% molybdenum. “MP20N” consists of 50% cobalt, 20% nickel, 20% chromium, and 10% molybdenum. Devices made from bioabsorbable or biostable polymers could also be used with the embodiments of the present invention.

Coating

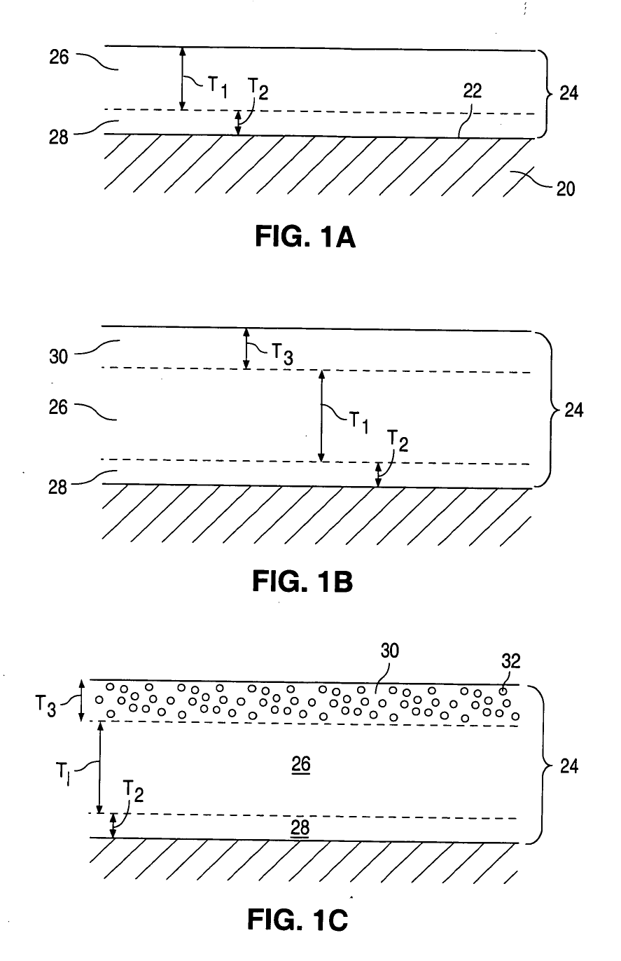

Referring to FIG. 1A, a body of a stent 20 is illustrated having a surface 22, e.g., a metallic surface such as stainless steel. A coating 24 is disposed on surface 22. Coating 24 includes a reservoir region 26 containing an active ingredient. A primer region 28 that is substantially free of any active ingredients is disposed underneath at least a portion of reservoir region 26. As illustrated in FIG. 1B, coating 24 can also include a barrier region 30 that is substantially free of any active ingredients. The Figures have not been drawn to scale, and the depth and thickness of the various regions and layers have been over or under emphasized for illustrative purposes.

In one embodiment of the present the invention, the interface between the primer region and the reservoir region is modified to increase the permeability of the primer layer to the active ingredient. The interface can be modified using one of several methods. These methods include, for example, partially blending of the polymers of the primer region with the polymers of the reservoir region, modifying the surface of the primer layer by forming areas of roughness, or forming a porous matrix on the surface of the primer layer, as more fully described below.

The interface between the primer region and the reservoir region can be modified by partially blending the polymers of the primer region with the polymers of the reservoir region during the coating process through the use of a common solvent. For example, primer region 28 can be formed on stent 20 by applying a primer coating composition having a polymer dissolved in a solvent. Stent 20 can then be baked to essentially remove the solvent in the composition to form the coating. Subsequently, another composition is applied having a polymer, a solvent and an active ingredient dispersed therein. If the solvent in the active ingredient composition is capable of dissolving the polymer in the primer layer, then the polymers of the primer region and the reservoir region will intermix or blend. This blending can allow the active ingredient to be absorbed or flux into primer layer 28.

In another embodiment, asperities, or areas of roughness, are formed on the surface of the primer layer to increase the permeability of the primer layer. The asperities enable the primer region to physically entrap the reservoir region. In addition, the asperities can promote the migration of the active ingredient from the reservoir region to the primer region through capillary action. Because the active ingredient migrates into the deeper portions of the coating, the diffusion rate of the active ingredient from the coating is decreased when the coated device is inserted into a body of a patient. As a result, the asperities can promote an increased residence time of the active ingredient and thereby prevent the “burst effect” of the active ingredient from the coating. “Burst effect” refers to the quick release of an active ingredient from a polymeric coating when the device is inserted into a biological lumen.

The primer region can include a porous matrix extending from the interface of the primer region and the reservoir region into the primer region. The porous matrix can extend partially into the primer region, or all the way up to the surface of the implantable device. The active ingredient can migrate from the reservoir region by capillary action into the primer region.

In another embodiment of the present invention, the thickness of the primer region is increased relative to the thickness of the reservoir region. The thicker primer region can decrease the diffusion rate of the active ingredient from the coating. Additionally, by increasing the thickness of the primer region, the primer region can act as a more effective tie layer between the surface of the device and the reservoir region. For example, the thicker primer region can reduce or prevent the formation of cracks in the stent coating as the stent is expanded. Typically the presence of an active ingredient in a polymeric matrix interferes with the ability of the matrix to adhere effectively to the surface of the device. An increase in the quantity of the active ingredient reduces the effectiveness of the adhesion. High drug loadings of, for example, 10-40% by weight in the coating significantly hinder the retention of the coating on the surface of the device. “Drug loading” means the percentage ratio of active ingredient to polymer by weight. By increasing the thickness of primer region 28 relative to reservoir region 26, primer region 28 can allow for the quantity of the active ingredient in reservoir region 26 to be increased without compromising the ability of reservoir region 26 to be effectively contained on the device during delivery and, if applicable, expansion of the device. For instance, the coating of the present invention has superior results when the drug loading is relatively high. In particular, the mechanical integrity of the coating of the present invention can withstand expansion of the stent (i.e., the coating does not significantly peel or crack) even when the drug loading in the coating is relatively high. In one embodiment of the present invention, drug loadings equal to or greater than 30% can be achieved.

Referring to FIG. 1B, by way of example, reservoir region 26 for coating 24 can have a thickness T₁ of about 0.5 microns to about 10 microns. Primer region 28 can have a thickness T₂, examples of which can be in the range of about 0.1 to about 10 microns, more narrowly about 0.5 to about 5 microns. The thickness of the reservoir region T₁ is measured from the outer surface of the reservoir region to the primer region prior to the migration of the active ingredient from the reservoir region to the primer region. Similarly, the thickness of the primer region T₂ is measured from the outer surface of the primer region to the surface of the stent prior to the migration of the active ingredient from the reservoir region to the primer region. In an embodiment of the present invention, T₂/T₁ is greater than or equal to 0.25. In another embodiment, T₂/T₁ is greater than or equal to 0.33. The particular thicknesses T₁ and T₂ are based in part on the type of procedure for which stent 20 is employed and the amount of the active ingredient that is desired to be delivered.

In some invention embodiments, T₂/T₁ can be 0.01 to 0.2; 0.05 to 0.2; 0.01 to 20; 0.05 to 20; 0.01 to 1; 0.05 to 1; or 0.01 to 0.05.

Alternatively, in some embodiments, reservoir region 26 for coating 24 can have a mass W₁. Primer region 28 can have a mass W₂. The mass of the reservoir region W₁ is exclusive of the active ingredient. In an embodiment of the present invention, W₂/W₁ is greater than or equal to 0.25. In another embodiment, W₂/W₁ is greater than or equal to 0.33. The particular mass W₁ and W₂ are based in part on the type of procedure for which stent 20 is employed and the amount of the active ingredient that is desired to be delivered.

In some invention embodiments, W₂/W₁ can be 0.01 to 0.2; 0.05 to 0.2; 0.01 to 20; 0.05 to 20; 0.01 to 1; 0.05 to 1; or 0.01 to 0.05.

Alternatively, in some embodiments, reservoir region 26 for coating 24 can have a mass WA₁. Primer region 28 can have a mass W₂. The mass of the reservoir region WA₁ includes the mass of the active ingredient. In an embodiment of the present invention, W₂/WA₁ is greater than or equal to 0.25. In another embodiment, W₂/WA₁ is greater than or equal to 0.33. The particular mass WA₁ and W₂ are based in part on the type of procedure for which stent 20 is employed and the amount of the active ingredient that is desired to be delivered.

In some invention embodiments, W₂/WA₁ can be 0.01 to 0.2; 0.05 to 0.2; 0.01 to 20; 0.05 to 20; 0.01 to 1; 0.05 to 1; or 0.01 to 0.05.

Referring to FIG. 1B, diffusion barrier region 30 can have any suitable thickness T₃, as the thickness T₃ is dependent on parameters such as, but not limited to, the desired rate or duration of release and the procedure for which stent 20 will be used. Diffusion barrier region 30 can have a thickness T₃ of about 0.1 to about 10 microns, more narrowly from about 0.25 to about 2 microns. Additionally, referring to FIG. 1C, barrier layer 30 can contain particles 32 to reduce the rate of release of the active ingredient from the coating. If particles are employed, for a smooth outer surface, the size of particles should not be greater than about 10% of thickness T₃ of diffusion barrier region 30. Additionally, the particle volume fraction Xₚ should not exceed about 0.74. Packing density or particle volume fraction Xₚ can be defined by the following equation:
Xₚ=V_{particies}/(V_{particles}+V_{polymer})

wherein V is volume.

Each of the layers of the polymeric coating can have different sections with different properties in order to provide a coating with variable active ingredient release parameters. As illustrated in FIG. 1D, for example, reservoir region 26 can include first and second reservoir sections 26A and 26B, each containing a different active ingredient, e.g., actinomycin D and taxol, respectively. Accordingly, coating 24 can carry a combination of at least two different active ingredients for sustained delivery. First and second sections 26A and 26B can be deposited by, for example, masking the area of primer region 28 over second section 26B and applying a first composition containing a first active ingredient to form first section 26A. First section 26A can then be masked and a second composition containing a second active ingredient can be applied to form second section 26B. This procedure can be followed to form any suitable number of sections containing a different active ingredient.

Barrier region 30 can be formed on reservoir sections 26A and 26B, as illustrated in FIG. 1D. Referring to FIG. 1E, barrier region 30 can also include a first barrier section 30A disposed over first reservoir section 26A containing a first active ingredient, e.g., actinomycin D. A second barrier section 30B can be formed over second reservoir section 26B containing a second active ingredient, e.g., taxol. First barrier section 30A is particle free and second barrier section 30B contains particles 32. As a result, coating 24 harbors two different release parameters for each of the active ingredients contained in reservoir sections 26A and 26B.

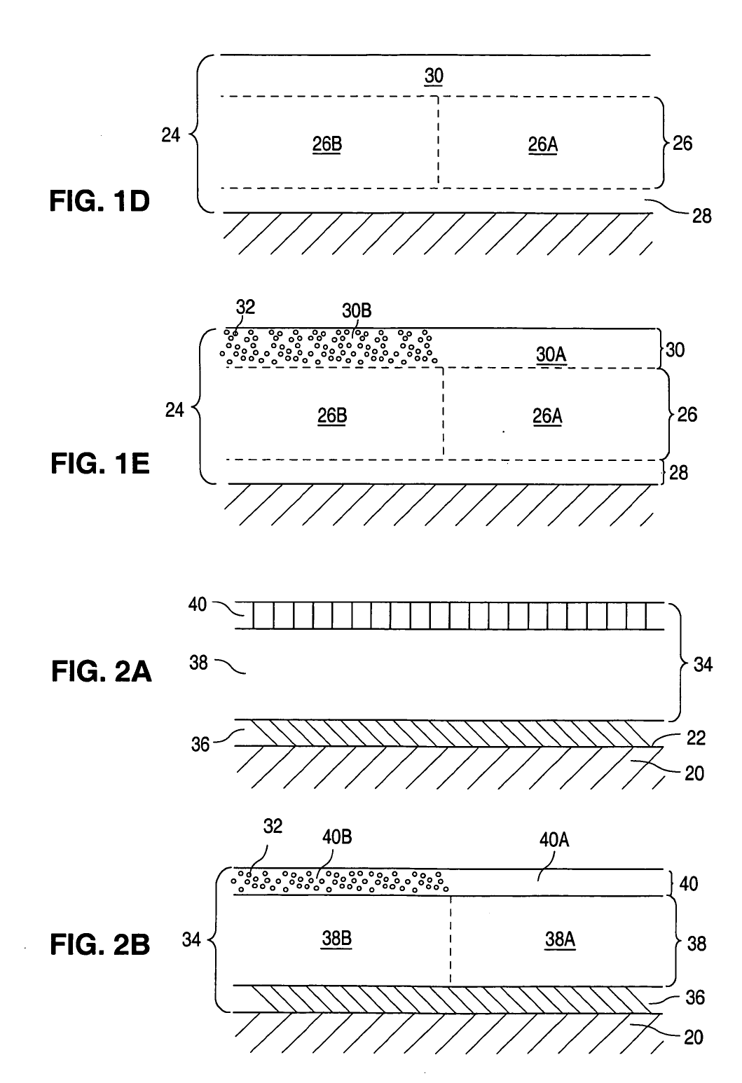

Different polymeric materials having interfacial compatibilities can be used to form individual, distinct layers for the primer, reservoir, and diffusion barrier components of the coating. Referring to FIG. 2A, a coating 34 is provided having a primer region 36, made from a first polymeric material, formed on surface 22 of stent 20. A reservoir region 38 made from a second polymeric material is deposited on a selected area of primer region 36. A barrier region 40, made from a third polymeric material can be deposited on reservoir region 38. Examples of different polymeric materials having interfacial compatibilities include, for example, an EVAL primer with a reservoir layer of ethylene vinylacetate; a poly(n-butyl methacrylate) primer with an EVAL reservoir layer; an EVAL primer and a reservoir layer of polycaprolactone; and an epoxy primer consisting of the diglycidylether of bisphenol A cured with polyamine curatives with an EVAL reservoir layer. Other combinations can be derived by one of ordinary skill in the art.

One of ordinary skill in the art can appreciate that a variety of coating combinations can be provided. For example, as illustrated in FIG. 2B, coating 34 contains primer region 36 made from a first polymeric material. Reservoir region 38, made from a second polymeric material, is formed on primer region 36. Reservoir region 38 contains first and second sections, illustrated as 38A and 38B. First and second sections 38A and 38B each contain a different active ingredient. Barrier region 40, made from a third polymeric material, can be deposited on reservoir region 38. Barrier region 40 includes a first section 40A deposited over first section 38A of reservoir region 38. Barrier region 40 additionally includes a second section 40B deposited over second section 38B of reservoir region 38. Second section 40B can include particles 32 and/or be made out of a fourth polymeric material to create a variety of different release parameters.

Composition for the Primer Layer

The embodiments of the composition for a primer layer are prepared by conventional methods wherein all components are combined, then blended. More particularly, a predetermined amount of a polymer or a prepolymer is added to a predetermined amount of a solvent or a combination of solvents. The mixture can be prepared in ambient pressure and under anhydrous atmosphere. If necessary, a free radical or UV initiator can be added to the composition for initiating the curing or crosslinking of the prepolymer. Heating and stirring and/or mixing can be employed to effect dissolution of the polymer into the solvent.

“Polymer,” “poly,” and “polymeric” are defined as compounds that are the product of a polymerization reaction and are inclusive of homopolymers, copolymers, terpolymers etc., including random, alternating, block, and graft variations thereof. The polymers should have a high capacity of adherence to the surface of an implantable device, such as a metallic surface of a stent. Stainless steel, such as 316L, is a commonly used material for the manufacturing of a stent. Stainless steel includes a chromium oxide surface layer which makes the stent corrosion resistant and confers, in large part, biocompatibility properties to the stent. The chromium oxide layer presents oxide, anionic groups, and hydroxyl moieties, which are polar. Consequently, polymeric materials with polar substituents and cationic groups can adhere to the surface. Representative examples of suitable polymeric material include polyisocyanates, unsaturated polymers, high amine content polymers, acrylates, polymers with high content of hydrogen bonding groups, silane coupling agents, titanates and zirconates.

Representative examples of polyisocyanates include triisocyanurate, alphatic polyisocyanate resins based on hexamethylene diisocyanate, aromatic polyisocyanate prepolymers based on diphenylmethane diisocyanate, polyisocyanate polyether polyurethanes based on diphenylmethane diisocyanate, polymeric isocyanates based on toluene diisocyanate, polymethylene polyphenyl isocyanate, and polyester polyurethanes.

Representative examples of unsaturated polymers include polyester diacrylates, polycaprolactone diacrylates, polyester diacrylates, polytetramethylene glycol diacrylate, polyacrylates with at least two acrylate groups, polyacrylated polyurethanes, and triacrylates. With the use of unsaturated prepolymers a free radical or UV initiator can be added to the composition for the thermal or UV curing or crosslinking process. For thermal curing, examples of free radicals initiators are benzoyl peroxide; bis(2,4-dichlorobenzoyl) peroxide; dicumyl peroxide; 2,5-bis(tert-butyl peroxy)-2,5-dimethyl hexane; ammonium persulfate, and 2,2′-azobisisobutyronitrile. As is understood by one of ordinary skill in the art, each initiator requires a different temperature to induce decomposition. For UV curing, examples of initiators include 2,2-dimethoxy-2-phenylacetophenone; 1-hydroxycyclohexyl phenyl ketone; benzoin ethyl ether; and benzophenone. These initiators can be activated by illumination with a medium pressure Hg bulb that contains wavelengths between 250 and 350 nm.

Representative examples of high amine content polymers include polyethyleneamine, polyallylamine, and polylysine.

Representative examples of acrylates include copolymers of ethyl acrylate, methyl acrylate, methacrylic acid, acrylic acid, and cyanoacrylates.

Representative examples of high content of hydrogen bonding group polymers include polyethylene-co-polyvinyl alcohol, epoxy polymers based on the diglycidylether of bisphenol A with amine crosslinking agents, epoxy polymers cured by polyols and lewis acid catalysts, epoxy phenolics, epoxy-polysulfides, ethylene vinyl acetate, melamine formaldehydes, polyvinylalcohol-co-vinyl acetate polymers, resorcinol-formaldehydes, urea-formaldehydes, polyvinylbutyral, polyvinylacetate, alkyd polyester resins, acrylic acid modified ethylene vinyl acetate polymers, methacrylic acid modified ethylene vinyl acetate polymers, acrylic acid modified ethylene acrylate polymers, methacrylic acid modified ethylene acrylate polymers, anhydride modified ethylene acrylate copolymers, and anhydride modified ethylene vinyl acetate polymers.

Representative examples of silane coupling agents include 3-aminopropyltriethoxysilane and (3-glydidoxypropyl) methyldiethoxysilane.

Representative examples of titanates include tetra-iso-propyl titanate and tetra-n-butyl titanate.

Representative examples of zirconates include n-propyl zirconate and n-butyl zirconate.

Biocompatible polymers can also be used for the primer material. Examples of biocompatible primers include poly(hydroxyvalerate), poly(L-lactic acid), polycaprolactone, poly(lactide-co-glycolide), poly(hydroxybutyrate), poly(hydroxybutyrate-co-valerate), polydioxanone, polyorthoesters, polyanhydrides, poly(glycolic acid), poly(D,L-lactic acid), poly(glycolic acid-co-trimethylene carbonate), polyphosphoesters, polyphosphoester urethanes, poly(amino acids), cyanoacrylates, poly(trimethylene carbonates), poly(iminocarbonate), copoly(ether-esters) (e.g. PEO/PLA), polyalkylene oxalates, polyphosphazenes and biomolecules such as fibrin, fibrinogen, cellulose, starch, collagen and hyaluronic acid. Also, polyurethanes, silicones, and polyesters could be used and other polymers could also be used if they can be dissolved and cured or polymerized on the stent such as polyolefins, polyisobutylene and ethylene-alphaolefin copolymers; acrylic polymers and copolymers, vinyl halide polymers and copolymers, such as polyvinyl chloride; polyvinyl ethers, such as polyvinyl methyl ether; polyvinylidene halides, such as polyvinylidene fluoride and polyvinylidene chloride; polyacrylonitrile; polyvinyl ketones; polyvinyl aromatics, such as polystyrene; polyvinyl esters, such as polyvinyl acetate; copolymers of vinyl monomers with each other and olefins, such as ethylene-methyl methacrylate copolymers, acrylonitrile-styrene copolymers, ABS resins, and ethylene-vinyl acetate copolymers; polyamides, such as Nylon 66 and polycaprolactam; alkyd resins; polycarbonates; polyoxymethylenes; polyimides; polyethers; epoxy resins; rayon; rayon-triacetate, cellulose, cellulose acetate, cellulose butyrate; cellulose acetate butyrate; cellophane; cellulose nitrate; cellulose propionate; cellulose ethers; and carboxymethyl cellulose.

A representative example of a very suitable choice of polymer for the primer layer is poly(butyl methacrylate) (PBMA). Ethylene vinyl alcohol is also functionally a very suitable choice of polymer. Ethylene vinyl alcohol copolymer, commonly known by the generic name EVOH or by the trade name EVAL, refers to copolymers comprising residues of both ethylene and vinyl alcohol monomers. One of ordinary skill in the art understands that ethylene vinyl alcohol copolymer may also be a terpolymer so as to include small amounts of additional monomers, for example less than about five (5) mole percentage of styrenes, propylene, or other suitable monomers. In a useful embodiment, the copolymer comprises a mole percent of ethylene of from about 27% to about 47%. Typically, 44 mole percent ethylene is suitable. Ethylene vinyl alcohol copolymers are available commercially from companies such as Aldrich Chemical Company, Milwaukee, Wis., or EVOH Company of America, Lisle, Ill., or can be prepared by conventional polymerization procedures that are well known to one of ordinary skill in the art. The copolymer possesses good adhesive qualities to the surface of a stent, particularly stainless steel surfaces, and has illustrated the ability to expand with a stent without any significant detachment of the copolymer from the surface of the stent.

The solvent should be mutually compatible with the polymer and should be capable of placing the polymer into solution at the concentration desired in the solution. Useful solvents should also be able to expand the chains of the polymer for maximum interaction with the surface of the device, such as a metallic surface of a stent. Examples of solvent can include, but are not limited to, dimethylsulfoxide (DMSO), chloroform, water (buffered saline), xylene, acetone, methanol, ethanol, 1-propanol, tetrahydrofuran, 1-butanone, dimethylformamide, dimethylacetamide, cyclohexanone, ethyl acetate, methylethylketone, propylene glycol monomethylether, isopropanol, N-methylpyrrolidinone, toluene and mixtures thereof.

By way of example, and not limitation, the polymer can comprise from about 0.1% to about 35%, more narrowly about 2% to about 20% by weight of the total weight of the composition, and the solvent can comprise from about 65% to about 99.9%, more narrowly about 80% to about 98% by weight of the total weight of the composition. A specific weight ratio is dependent on factors such as the material from which the implantable device is made and the geometrical structure of the device.

A fluid can also be added to the composition to enhance the wetting of the composition for a more uniform coating application. To enhance the wetting of the composition, a suitable fluid typically has a high capillary permeation. Capillary permeation or wetting is the movement of a fluid on a solid substrate driven by interfacial energetics. Capillary permeation is quantitated by a contact angle, defined as an angle at the tangent of a droplet in a fluid phase that has taken an equilibrium shape on a solid surface. A low contact angle means a higher wetting liquid. A suitably high capillary permeation corresponds to a contact angle less than about 90°. The wetting fluid, typically, should have a viscosity not greater than about 50 centipoise at room temperature, narrowly about 0.3 to about 5 centipoise, more narrowly about 0.4 to about 2.5 centipoise. The wetting fluid, accordingly, when added to the composition, reduces the viscosity of composition.

The wetting fluid should be mutually compatible with the polymer and the solvent and should not precipitate the polymer. The wetting fluid can also act as the solvent. Useful examples of the wetting fluid include, but are not limited to, tetrahydrofuran (THF), dimethylformamide (DMF), 1-butanol, n-butyl acetate, dimethyl acetamide (DMAC), and mixtures and combinations thereof. By way of example and not limitation, the polymer can comprise from about 0.1% to about 35%, more narrowly from about 2% to about 20% by weight of the total weight of the composition; the solvent can comprise from about 19.9% to about 98.9%, more narrowly from about 58% to about 84% by weight of the total weight of the composition; the wetting fluid can comprise from about 1% to about 80%, more narrowly from about 5% to about 40% by weight of the total weight of the composition. The specific weight ratio of the wetting fluid depends on the type of wetting fluid employed and type of and the weight ratio of the polymer and the solvent. More particularly, tetrahydrofuran used as the wetting fluid can comprise, for example, from about 1% to about 44%, more narrowly about 21% by weight of the total weight of the solution. Dimethylformamide used as the wetting fluid can comprise, for example, from about 1% to about 80%, more narrowly about 8% by weight of the total weight of the solution. 1-butanol used as the wetting fluid can comprise, for example, from about 1% to about 33%, more narrowly about 9% by weight of the total weight of the solution. N-butyl acetate used as the wetting fluid can comprise, for example, from about 1% to about 34%, more narrowly about 14% by weight of the total weight of the solution. Dimethyl acetamide used as the wetting fluid can comprise, for example, from about 1% to about 40%, more narrowly about 20% by weight of the total weight of the solution.

Table 1 illustrates some examples of suitable combinations for the primer composition:

TABLE 1

| Polymer | Solvent | Wetting Fluid | Initiators |
|---|---|---|---|
| EVAL | DMSO | — | — |
| EVAL | DMSO | THF | — |
| polyester polyurethanes | dimethylformamide | — | — |
| polyester polyurethanes | dimethylformamide | DMAC | — |
| polycaprolactone | Chloroform | n-butyl acetate | |
| polyacrylate polyurethane | ethyl acetate | — | benzophenone |
| polyacrylated | ethyl acetate | — | 1- |
| polyurethane | | | hydroxycyclohexyl |
| | | | phenyl ketone |
| polyethyleneamine | H₂O | — | — |
| methacrylic acid | THF | — | — |
| copolymer | | | |
| ethylene vinyl acetate | methylethylketone | — | — |
| (e.g., 40% vinyl acetate | | | |
| content) | | | |
| aminopropyl- | ethanol/water | — | — |
| triethoxysilane | 95/5 blend (w/w) | | |
| (3- | Toluene | — | — |
| glydidoxypropyl) | | | |
| methyldiethoxysilane | | | |
| tetra-iso-propyl | isopropanol | — | — |
| titanate | | | |
| (e.g., 0.25% w/w in isopropanol) | | | |
| tetra-n-butyl titanate | ethyl acetate | — | — |
| (e.g., 0.1-5% w/w in ethyl | | | |
| acetate) | | | |

Composition for the Active Ingredient Layer

The embodiments of the composition for an active ingredient-containing or reservoir layer are prepared by conventional methods wherein all components are combined, then blended. More particularly, a predetermined amount of a polymeric compound is added to a predetermined amount of a mutually compatible solvent or combination of solvents. The polymeric compound can be added at ambient pressure and under anhydrous atmosphere. If necessary, gentle heating and stirring and/or mixing can be employed to effect dissolution of the polymer into the solvent, for example 12 hours in a water bath at about 60° C.

The polymer chosen must be a polymer that is biocompatible and minimizes irritation to the vessel wall when the device is implanted. The polymer may be either a biostable or a bioabsorbable polymer. Bioabsorbable polymers that could be used include poly(hydroxyvalerate), poly(L-lactic acid), polycaprolactone, poly(lactide-co-glycolide), poly(hydroxybutyrate), poly(hydroxybutyrate-co-valerate), polydioxanone, polyorthoesters, polyanhydrides, poly(glycolic acid), poly(D,L-lactic acid), poly(glycolic acid-co-trimethylene carbonate), polyphosphoesters, polyphosphoester urethanes, poly(amino acids), cyanoacrylates, poly(trimethylene carbonate), poly(iminocarbonate), copoly(ether-esters) (e.g., PEO/PLA), polyalkylene oxalates, polyphosphazenes and biomolecules such as fibrin, fibrinogen, cellulose, starch, collagen and hyaluronic acid. Also, biostable polymers with a relatively low chronic tissue response such as polyurethanes, silicones, and polyesters could be used and other polymers could also be used if they can be dissolved and cured or polymerized on the stent such as polyolefins, polyisobutylene and ethylene-alphaolefin copolymers; acrylic polymers and copolymers, vinyl halide polymers and copolymers, such as polyvinyl chloride; polyvinyl ethers, such as polyvinyl methyl ether; polyvinylidene halides, such as polyvinylidene fluoride and polyvinylidene chloride; polyacrylonitrile; polyvinyl ketones; polyvinyl aromatics, such as polystyrene; polyvinyl esters, such as polyvinyl acetate; copolymers of vinyl monomers with each other and olefins, such as ethylene-methyl methacrylate copolymers, acrylonitrile-styrene copolymers, ABS resins, and ethylene-vinyl acetate copolymers; polyamides, such as Nylon 66 and polycaprolactam; alkyd resins; polycarbonates; polyoxymethylenes; polyimides; polyethers; epoxy resins; rayon; rayon-triacetate; cellulose, cellulose acetate, cellulose butyrate; cellulose acetate butyrate; cellophane; cellulose nitrate; cellulose propionate; cellulose ethers; and carboxymethyl cellulose.

Ethylene vinyl alcohol is functionally a very suitable choice of polymer. The copolymer allows for good control capabilities over the release rate of the active ingredient. As a general rule, an increase in the amount of the ethylene comonomer content decreases the rate that the active ingredient is released from the copolymer matrix. The release rate of the active ingredient typically decreases as the hydrophilicity of the copolymer decreases. An increase in the amount of the ethylene comonomer content increases the overall hydrophobicity of the copolymer, especially as the content of vinyl alcohol is concomitantly reduced. It is also known that the release rate and the cumulative amount of the active ingredient that is released is directly proportional to the total initial content of the ingredient in the copolymer matrix. Accordingly, a wide spectrum of release rates can be achieved by modifying the ethylene comonomer content and the initial amount of the active ingredient.

The choice of polymer for the reservoir layer can be the same as or different from the selected polymer for the primer layer. The use of the same polymer significantly reduces or eliminates any interfacial incompatibilities, such as lack of an adhesive tie or bond, which may exist with the employment of two different polymeric layers.

The solvent should be capable of placing the polymer into solution at the concentration desired in the solution. Examples of solvent can include, but are not limited to, DMSO, chloroform, water (buffered saline), xylene, acetone, methanol, ethanol, 1-propanol, tetrahydrofuran, 1-butanone, dimethylformamide, dimethylacetamide, cyclohexanone, and N-methylpyrrolidinone. With the use of low ethylene content, e.g., 29 mol %, ethylene vinyl alcohol copolymer, a suitable choice of solvent is iso-propylalcohol (IPA) admixed with water.

Sufficient amounts of an active ingredient are dispersed in the blended composition of the polymer and the solvent. The active ingredient should be in true solution or saturated in the blended composition. If the active ingredient is not completely soluble in the composition, operations including mixing, stirring, and/or agitation can be employed to effect homogeneity of the residues. The active ingredient may be added so that the dispersion is in fine particles. The mixing of the active ingredient can be conducted in an anhydrous atmosphere, at ambient pressure, and at room temperature such that supersaturating the active ingredient is not desired.

The active ingredient should inhibit the activity of vascular smooth muscle cells. More specifically, the active ingredient is aimed at inhibiting abnormal or inappropriate migration and/or proliferation of smooth muscle cells.

“Smooth muscle cells” include those cells derived from the medial and adventitial layers of the vessel which proliferate in intimal hyperplastic vascular sites following vascular trauma or injury. Under light microscopic examination, characteristics of smooth muscle cells include a histological morphology of a spindle shape with an oblong nucleus located centrally in the cell with nucleoli present and myofibrils in the sarcoplasm. Under electron microscopic examination, smooth muscle cells have long slender mitochondria in the juxtanuclear sarcoplasm, a few tubular elements of granular endoplasmic reticulum, and numerous clusters of free ribosomes. A small Golgi complex may also be located near one pole of the nucleus.

“Migration” of smooth muscle cells means movement of these cells in vivo from the medial layers of a vessel into the intima, such as may also be studied in vitro by following the motion of a cell from one location to another, e.g., using time-lapse cinematography or a video recorder and manual counting of smooth muscle cell migration out of a defined area in the tissue culture over time.

“Proliferation” of smooth muscle cells means increase in cell number.

“Abnormal” or “inappropriate” proliferation means division, growth or migration of cells occurring more rapidly or to a significantly greater extent than typically occurs in a normally functioning cell of the same type, i.e., hyperproliferation.

“Inhibiting” cellular activity means reducing, delaying or eliminating smooth muscle cell hyperplasia, restenosis, and vascular occlusions, particularly following biologically or mechanically mediated vascular injury or trauma or under conditions that would predispose a mammal to suffer such a vascular injury or trauma. As used herein, the term “reducing” means decreasing the intimal thickening that results from stimulation of smooth muscle cell proliferation. “Delaying” means retarding the progression of the hyper-proliferative vascular disease or delaying the time until onset of visible intimal hyperplasia, as observed, for example, by histological or angiographic examination. “Elimination” of restenosis following vascular trauma or injury means completely “reducing” and/or completely “delaying” intimal hyperplasia in a patient to an extent which makes it no longer necessary to surgically intervene, i.e., to re-establish a suitable blood flow through the vessel by, for example, repeat angioplasty, atherectomy, or coronary artery bypass surgery. The effects of reducing, delaying, or eliminating restenosis may be determined by methods known to one of ordinary skill in the art, including, but not limited to, angiography, intravascular ultrasound, fluoroscopic imaging, fiber optic visualization, optical coherence tomography, intravascular MRI, or biopsy and histology. Biologically mediated vascular injury includes, but is not limited to, injury caused by or attributed to autoimmune disorders, autoimmune related disorders, infectious disorders including endotoxins and herpes viruses such as cytomegalovirus, metabolic disorders such as atherosclerosis, and vascular injury resulting from hypothermia and irradiation. Mechanically mediated vascular injury includes, but is not limited to, vascular injury caused by catheterization procedures or vascular scraping procedures such as percutaneous transluminal coronary angioplasty, vascular surgery, stent placement, transplantation surgery, laser treatment, and other invasive procedures which disrupted the integrity of the vascular intima or endothelium. The active ingredient of the invention is not restricted in use for therapy following vascular injury or trauma; rather, the usefulness of the active ingredient will also be determined by the ingredient's ability to inhibit cellular activity of smooth muscle cells or inhibit the development of restenosis.

The active ingredient also includes any substance capable of exerting a therapeutic or prophylactic effect in the practice of the present invention as well as having positive pharmacological effects on the expression of the extracellular matrix. The active ingredient can also be for enhancing wound healing in a vascular site and improving the structural and elastic properties of the vascular site.

In these or other embodiments, the medical device further comprises a therapeutic agent. The following types of therapeutic agents are found in some invention embodiments: proteins, peptides, antiproliferatives, antineoplastics, antiinflammatories, antiplateletes, anticoagulants, antifibrins, antithrombins, antimitotics, antibiotics, antioxidants, or their mixtures.

Some embodiments add conventional drugs, such as small, hydrophobic drugs, to invention polymers (as discussed in any of the embodiments, above), making them biodegradable, drug systems. Some embodiments graft on conventional drugs or mix conventional drugs with invention polymers. Invention polymers can be coated as blends with a variety of biobeneficial polymers. Moreover, they can serve as base or topcoat layers for biobeneficial polymer layers.

The bioactive agents can be any moiety capable of contributing to a therapeutic effect, a prophylactic effect, both a therapeutic and prophylactic effect, or other biologically active effect in a mammal. The agent can also have diagnostic properties. The bioactive agents include, but are not limited to, small molecules, nucleotides, oligonucleotides, polynucleotides, amino acids, oligopeptides, polypeptides, and proteins. In one example, the bioactive agent inhibits the activity of vascular smooth muscle cells. In another example, the bioactive agent controls migration or proliferation of smooth muscle cells to inhibit restenosis.

Bioactive agents include, but are not limited to, antiproliferatives, antineoplastics, antimitotics, anti-inflammatories, antiplatelets, anticoagulants, antifibrins, antithrombins, antibiotics, antiallergics, antioxidants, and any prodrugs, metabolites, analogs, homologues, congeners, functional derivatives, structural derivatives, salts and combinations thereof. It is to be appreciated that one skilled in the art should recognize that some of the groups, subgroups, and individual bioactive agents may not be used in some embodiments of the present invention.

Examples of suitable therapeutic and prophylactic agents include synthetic inorganic and organic compounds, proteins and peptides, polysaccharides and other sugars, lipids, and DNA and RNA nucleic acid sequences having therapeutic, prophylactic or diagnostic activities. Nucleic acid sequences include genes, antisense molecules that bind to complementary DNA to inhibit transcription, and ribozymes. Some other examples of other bioactive agents include antibodies, receptor ligands, enzymes, adhesion peptides, blood clotting factors, inhibitors or clot dissolving agents such as streptokinase and tissue plasminogen activator, antigens for immunization, hormones and growth factors, oligonucleotides such as antisense oligonucleotides and ribozymes and retroviral vectors for use in gene therapy.

Antiproliferatives include, for example, actinomycin D, actinomycin IV, actinomycin I₁, actinomycin X₁, actinomycin C₁, and dactinomycin (COSMEGEN®, Merck & Co., Inc.).

Antineoplastics or antimitotics include, for example, paclitaxel (TAXOL®, Bristol-Myers Squibb Co.), docetaxel (TAXOTERE®, Aventis S. A.), methotrexate, azathioprine, vincristine, vinblastine, fluorouracil, adriamycin, mutamycin, doxorubicin hydrochloride (ADRIAMYCIN®, Pfizer, Inc.) and mitomycin (MUTAMYCIN®, Bristol-Myers Squibb Co.), and any prodrugs, metabolites, analogs, homologues, congeners, functional derivatives, structural derivatives, salts and combinations thereof.

Antiplatelets, anticoagulants, antifibrin, and antithrombins include, for example, aspirin, sodium heparin, low molecular weight heparins, heparinoids, hirudin, argatroban, forskolin, vapiprost, prostacyclin and prostacyclin analogues, dextran, D-phe-pro-arg-chloromethylketone (synthetic antithrombin), dipyridamole, glycoprotein IIb/IIIa platelet membrane receptor antagonist antibody, recombinant hirudin, and thrombin inhibitors (ANGIOMAX®, Biogen, Inc.), and any prodrugs, metabolites, analogs, homologues, congeners, functional derivatives, structural derivatives, salts and combinations thereof.

Cytostatic or antiproliferative agents include, for example, angiopeptin, angiotensin converting enzyme inhibitors such as captopril (CAPOTEN® and CAPOZIDE®, Bristol-Myers Squibb Co.), cilazapril or lisinopril (PRINIVIL® and PRINZIDE®, Merck & Co., Inc.); calcium channel blockers such as nifedipine; colchicines; fibroblast growth factor (FGF) antagonists, fish oil (omega 3-fatty acid); histamine antagonists; lovastatin (MEVACOR®, Merck & Co., Inc.); monoclonal antibodies including, but not limited to, antibodies specific for Platelet-Derived Growth Factor (PDGF) receptors; nitroprusside; phosphodiesterase inhibitors; prostaglandin inhibitors; suramin; serotonin blockers; steroids; thioprotease inhibitors; PDGF antagonists including, but not limited to, triazolopyrimidine; and nitric oxide, and any prodrugs, metabolites, analogs, homologues, congeners, functional derivatives, structural derivatives, salts and combinations thereof.

Antiallergic agents include, but are not limited to, pemirolast potassium (ALAMAST®, Santen, Inc.), and any prodrugs, metabolites, analogs, homologues, congeners, functional derivatives, structural derivatives, salts and combinations thereof.

Examples of anti-inflammatory agents including steroidal and nonsteroidal anti-inflammatory agents include tacrolimus, dexamethasone, clobetasol, combinations thereof.

Other bioactive agents useful in the present invention include, but are not limited to, free radical scavengers; nitric oxide donors; rapamycin; methyl rapamycin; 42-Epi-(tetrazoylyl) rapamycin (ABT-578); everolimus; tacrolimus; 40-O-(2-hydroxy)ethyl-rapamycin; 40-O-(3-hydroxy)propyl-rapamycin; 40-O-[2-(2-hydroxy)ethoxy]-ethyl-rapamycin; tetrazole containing rapamycin analogs such as those described in U.S. Pat. No. 6,329,386; estradiol; clobetasol; idoxifen; tazarotene; alphainterferon; host cells such as epithelial cells; genetically engineered epithelial cells; dexamethasone; and any prodrugs, metabolites, analogs, homologues, congeners, functional derivatives, structural derivatives, salts and combinations thereof.

Free radical scavengers include, but are not limited to, 2,2′,6,6′-tetramethyl-1-piperinyloxy, free radical (TEMPO); 4-amino-2,2′,6,6′-tetramethyl-1-piperinyloxy, free radical (4-amino-TEMPO); 4-hydroxy-2,2′,6,6′-tetramethylpiperidene-1-oxy, free radical (4-hydroxy-TEMPO), 2,2′,3,4,5,5′-hexamethyl-3-imidazolinium-1-yloxy methyl sulfate, free radical; 4-carboxy-2,2′,6,6′-tetramethyl-1-piperinyloxy, free radical (4-carboxy-TEMPO); 16-doxyl-stearic acid, free radical; superoxide dismutase mimic (SODm) and any analogs, homologues, congeners, functional derivatives, structural derivatives, salts and combinations thereof.

Nitric oxide donors include, but are not limited to, S-nitrosothiols, nitrites, N-oxo-N-nitrosamines, substrates of nitric oxide synthase, diazenium diolates such as spermine diazenium diolate and any analogs, homologues, congeners, functional derivatives, structural derivatives, salts and combinations thereof.

Other therapeutic substances or agents which may be appropriate include imatinib mesylate, pimecrolimus, and midostaurin.

Other therapeutic substances or agents which may be appropriate include alpha-interferon, bioactive RGD, and genetically engineered epithelial cells.

The foregoing substances can also be used in the form of prodrugs or co-drugs thereof. The foregoing substances are listed by way of example and are not meant to be limiting. Other active agents which are currently available or that may be developed in the future are equally applicable.

The dosage or concentration of the bioactive agent required to produce a favorable therapeutic effect should be less than the level at which the bioactive agent produces toxic effects and greater than the level at which non-therapeutic results are obtained. The dosage or concentration of the bioactive agent can depend upon factors such as the particular circumstances of the patient; the nature of the trauma; the nature of the therapy desired; the time over which the ingredient administered resides at the vascular site; and if other active agents are employed, the nature and type of the substance or combination of substances. Therapeutic effective dosages can be determined empirically, for example by infusing vessels from suitable animal model systems and using immunohistochemical, fluorescent or electron microscopy methods to detect the agent and its effects, or by conducting suitable in vitro studies. Standard pharmacological test procedures to determine dosages are understood by one of ordinary skill in the art.

Examples of diagnostic agents include radioopaque materials and include, but are not limited to, materials comprising iodine or iodine-derivatives such as, for example, iohexal and iopamidol, which are detectable by x-rays. Other diagnostic agents such as, for example, radioisotopes, are detectable by tracing radioactive emissions. Other diagnostic agents may include those that are detectable by magnetic resonance imaging (MRI), ultrasound and other imaging procedures such as, for example, fluorescence and positron emission tomagraphy (PET). Examples of agents detectable by MRI are paramagnetic agents, which include, but are not limited to, gadolinium chelated compounds. Examples of agents detectable by ultrasound include, but are not limited to, perflexane. Examples of fluorescence agents include, but are not limited to, indocyanine green. Examples of agents used in diagnostic PET include, but are not limited to, fluorodeoxyglucose, sodium fluoride, methionine, choline, deoxyglucose, butanol, raclopride, spiperone, bromospiperone, carfentanil, and flumazenil.

Some embodiments choose the drug such that it does not contain at least one of or any combination of antiproliferative, antineoplastic, antiinflammatory, antiplatelet, anticoagulant, antifibrin, antithrombin, antimitotic, antibiotic, or antioxidant substances. Some invention embodiments choose the drug such that it does not contain at least one of or any combination of actinomycin D, derivatives and analogs of Actinomycin D, dactinomycin, actinomycin IV, actinomycin I₁, actinomycin X₁, actinomycin C₁, paclitaxel, docetaxel, aspirin, sodium heparin, low molecular weight heparin, hirudin, argatroban, forskolin, vapiprost, prostacyclin, prostacyclin analogs, dextran, D-phe-pro-arg-chloromethylketone (synthetic antithrombin), dipyridamole, glycoprotein IIb/IIIa platelet membrane receptor antagonist, recombinant hirudin, thrombin inhibitor and 7E-3B, methotrexate, azathioprine, vincristine, vinblastine, fluorouracil, adriamycin, mutamycin, angiopeptin, angiotensin converting enzyme inhibitors, CAPTOPRIL, CILAZAPRIL, or LISINOPRIL, calcium channel blockers, Nifedipine, colchicine, fibroblast growth factor (FGF) antagonists, histamine antagonist, LOVASTATIN, monoclonal antibodies, PDGF receptors, nitroprusside, phosphodiesterase inhibitors, prostaglandin inhibitor, Seramin, PDGF antagonists, serotonin blockers, thioprotease inhibitors, triazolopyrimidine, nitric oxide, alphainterferon, genetically engineered epithelial cells, dexamethasone, estradiol, clobetasol prop ionate, cisplatin, insulin sensitizers, receptor tyro sine kinase inhibitors, carboplatin, rapamycin, 40-O-(2-hydroxy)ethyl-rapamycin, or a functional analogs of 40O-(2-hydroxy)ethyl-rapamycin, structural derivative of 40-O-(2-hydroxy)ethylrapamycin, 40-O-(3-hydroxy)propyl-rapamycin, and 40-O-2-(2-hydroxy)ethoxyethylrapamycin.

Some invention embodiments comprise a drug or drug combination, and some require a drug or combination of drugs. Of the drugs specifically listed above, some invention embodiments exclude a single or any combination of these drugs.

The dosage or concentration of the active ingredient required to produce a favorable therapeutic effect should be less than the level at which the active ingredient produces toxic effects and greater than the level at which non-therapeutic results are obtained. The dosage or concentration of the active ingredient required to inhibit the desired cellular activity of the vascular region can depend upon factors such as the particular circumstances of the patient; the nature of the trauma; the nature of the therapy desired; the time over which the ingredient administered resides at the vascular site; and if other bioactive substances are employed, the nature and type of the substance or combination of substances. Therapeutic effective dosages can be determined empirically, for example by infusing vessels from suitable animal model systems and using immunohistochemical, fluorescent or electron microscopy methods to detect the agent and its effects, or by conducting suitable in vitro studies. Standard pharmacological test procedures to determine dosages are understood by one of ordinary skill in the art.

By way of example, the polymer can comprise from about 0.1% to about 35%, more narrowly from about 2% to about 20% by weight of the total weight of the composition, the solvent can comprise from about 59.9% to about 99.8%, more narrowly from about 79% to about 87% by weight of the total weight of the composition, and the active ingredient can comprise from about 0.1% to about 75%, more narrowly from about 20% to about 60% by weight of the total weight of the composition. Selection of a specific weight ratio of the polymer and solvent is dependent on factors such as, but not limited to, the material from which the device is made, the geometrical structure of the device, and the type and amount of the active ingredient employed. The particular weight percentage of the active ingredient mixed within the composition depends on factors such as duration of the release, cumulative amount of release, and release rate that is desired.

Optionally, a second fluid or solvent, such as tetrahydrofuran (THF) or dimethylformamide (DMF) can be used to improve the solubility of an active ingredient in the composition and/or to increase the wetting of the composition. Increasing the wetting of the composition has been discovered to lead to the application of a more uniformed coating. The second fluid or solvent can be added to the composition or the active ingredient can be added to the second solvent prior to admixture with the blend.

With use of a second fluid, by way of example, the polymer can comprise from about 0.1% to about 35%, more narrowly from about 2% to about 20% by weight of the total weight of the composition, the solvent can comprise from about 19.8% to about 98.8%, more narrowly from about 49% to about 79% by weight of the total weight of the composition, the second solvent can comprise from about 1% to about 80%, more narrowly from about 5% to about 40% by weight of the total weight of the composition, and the active ingredient can comprise from about 0.1% to about 40%, more narrowly from about 1% to about 9% by weight of the total weight of the composition. Selection of a specific weight ratio of the polymer, the solvent, and the second solvent is dependent on factors such as, but not limited to, the material from which the implantable device is made, the geometrical structure of the device, and the type and amount of the active ingredient employed. The particular weight percentage of the active ingredient mixed within the composition depends on factors such as duration of the release, cumulative amount of release, and release rate that is desired.

Table 2 is an exemplary list of suitable combinations:

TABLE 2

| | | SECOND | ACTIVE |
|---|---|---|---|
| POLYMER | SOLVENT | SOLVENT | INGREDIENT |
| EVAL (29 mol % | IPA/H₂O | — | Actinomycin D |
| ethylene content e.g., | (1:1) | | |
| Soarnol ®) | | | |
| EVAL (44 mol % | DMSO | THF | Actinomycin D |
| ethylene content) | | | |
| EVAL | DMSO | THF | Actinomycin D |
| EVAL | DMSO | DMF | Paclitaxel |
| poly(L-lactic acid) | chloroform | — | dexamethasone |
| poly(lactic acid-co- | acetone | — | dexamethasone |
| glycolic acid) | | | |
| Polyether urethane | N-methyl | — | tocopherol |
| | pyrrolidinone | | |

Composition for the Rate Reducing Membrane

The embodiments of the composition for a rate-reducing membrane or diffusion barrier layer are prepared by conventional methods wherein all components are combined. In the embodiment with the use of particles, dispersion techniques should also be employed to circumvent agglomeration or formation of particle flocs.

More particularly, the composition for the barrier layer can be applied on a selected portion of the reservoir layer. The barrier layer can reduce the rate of release or delay the time at which the active ingredient is released from the reservoir region. In one embodiment, for maximum blood compatibility, polyethylene glycol or polyethylene oxide can also be added to the blend. Ethylene vinyl alcohol is functionally a very suitable choice of polymer. The copolymer allows for good control capabilities over the release rate of the active ingredient. As a general rule, an increase in the amount of the ethylene comonomer content decreases the rate that the active ingredient is released from the copolymer matrix. The release rate of the active ingredient decreases as the hydrophilicity of the polymer decreases. An increase in the amount of the ethylene comonomer content increases the overall hydrophobicity of the copolymer, especially as the content of vinyl alcohol is concomitantly reduced.

Usefully, the choice of polymer for the barrier layer can be the same as the selected polymer for the reservoir. The use of the same polymer can significantly reduce or eliminate interfacial incompatibilities, such as lack of adhesion, which may exist in the employment of two different polymeric layers.

Particles of inorganic or organic type can be added to the blend. The particles can be made from any suitable material having barrier-type properties, such as, but not limited to tortuousity, excluded volume, and adsorptivity. “Tortuosity” refers to the exclusion of space in the polymer matrix for the creation of a defined space or a tortuous path through and about which the active ingredient must travel to be expelled from the layer. “Excluded volume” refers to the volume displaced by the particles that would otherwise be available for the diffusion of the active ingredient. “Adsorptivity” refers to the chromatographic effect which is dependent upon the interaction between the active ingredient used in combination with the particle. The active ingredient may be partially adsorbed and released by the surface of the particles, such as silica or fumed carbon particles.

The particles should be dispersed in the blend. “Dispersed” is defined as the particles being present as individual particles, not agglomerates or flocs. In certain polymer-solvent blends, certain particles will disperse with ordinary mixing. Otherwise the particles can be dispersed in the composition by high shear processes such as ball mill, disc mill, sand mill, attritor, rotor stator mixer, ultrasonication—all such high shear dispersion techniques being well known to one of ordinary skill in the art. Optionally, one of the aforementioned wetting fluids can also be added to the blend. The wetting fluid can be added prior to, contemporaneously with, or subsequent to the agitation. Biocompatible dispersing agents in the form of surfactants, emulsifiers, or stabilizers may also be added to the blend to assist in particle dispersion.

The particles can be made from a metal oxide, such as rutile titanium oxide, anatase titanium dioxide, niobium oxide, tantalum oxide, zirconium oxide, iridium oxide, or tungsten oxide. In another embodiment, the particles can be made from a main group oxide such as silica (silicon oxide) or alumina (aluminum oxide). Metallic particles such as gold, hafnium, platinum, iridium, palladium, tungsten, tantalum, niobium, zirconium, titanium, aluminum, or chromium can also be employed. In another embodiment, carbonaceous particles made from, for example, lamp black, furnace black, carbon black, fumed carbon black, gas black, channel black, activated charcoal, diamond, diamond like carbon, or CVD diamond can be employed. In yet another embodiment, the particles can be made from nitrides such as titanium nitride, chromium nitride, and zirconium nitride. In yet another embodiment, carbides such as tungsten carbide, silicon carbide, or titanium carbide, and calcium salts such as hydroxyapatite, dahlite, brushite, tricalcium phosphate, calcium sulphate, and calcium carbonate can be used. Other inorganic particles can include particles made from silicides, barium titanate, and strontium titanate.

The particles can also be made from a suitable polymer including polymers of polyolefins, polyurethanes, cellulosics (i.e., polymers having mer units derived from cellulose), polyesters, polyamides, poly(hexamethylene isophthalamide/terephthalamide) (commercially available as SELAR PA™), poly(ethylene terephthalate-co-p-oxybenzoate) (PET/PHB, e.g., copolymer having about 60-80 mole percent PHB), poly(hydroxy amide ethers), polyacrylates, polyacrylonitriie, acrylonitrile/styrene copolymer (commercially available as LOPAC), rubber-modified acrylonitrile/acrylate copolymer (commercially available as BAREX), poly(methyl methacrylate), liquid crystal polymers (LCP) (e.g., VECTRA available from Hoescht-Celanese, ZENITE available from DuPont, and XYDAR available from Amoco Performance Chemicals), poly(phenylene sulfide), polystyrenes, polycarbonates, poly(vinyl alcohols), poly(ethylene-vinyl alcohol) (EVAL, e.g., having about 27 to about 47 mole percent of ethylene content), epoxies composed of bisphenol A based diepoxides with amine cure, aliphatic polyketones (e.g., CARILON available from Shell, and KETONEX available from British Petroleum), polysulfones, poly(estersulfone), poly(urethane-sulfone), poly(carbonate-sulfone), poly(3-hydroxyoxetane), poly(amino ethers), gelatin, amylose, parylene-C, parylene-D, parylene-N.

Representatives polyolefins include those based upon alpha-monoolefin monomers having from about 2 to 6 carbon atoms and halogen substituted olefins, i.e., halogenated polyolefins. By way of example, and not limitation, low to high density polyethylenes, essentially unplasticized poly (vinyl chloride), poly (vinylidene chloride), poly (vinyl fluoride), poly (vinylidene fluoride), poly (tetrafluoroethylene) (Teflon), poly (chlorotrifluoroethylene) (KEL-F), and mixtures thereof are suitable. Low to high density polyethylenes are generally understood to have densities of about 0.92 g cm⁻³ to about 0.96 g cm⁻³, however, no bright line can be drawn for density classifications and the density can vary according to the supplier.

Representative polyurethanes include polyurethanes having a glass transition temperature above a storage or ambient temperature, for example having a glass transition temperature of at least 40° C. to 60° C., or having a non-polar soft segment which includes a hydrocarbon, silicone, fluorosilicone, or mixtures thereof. For example, ELAST-EON, manufactured by Elastomedic/CSIRO Molecular Science, is a polyurethane with a non-polar soft segment which is made from 1,4-butanediol, 4,4′-methylenediphenyl diisocyanate, and a soft segment composed of a blend poly(hexamethylene oxide) (PHMO) and bishydroxyethoxypropylpolydimethylsiloxane (PDMS). A useful example has a blend of 20% by weight PHMO and 80% by weight PDMS.

Representative examples of cellulosics include, but are not limited to, cellulose acetate having a degree of substitution (DS) greater than about 0.8 or less than about 0.6, ethyl cellulose, cellulose nitrate, cellulose acetate butyrate, methyl cellulose, and mixtures thereof.

Representative polyesters include saturated or unsaturated polyesters such as, but not limited to, poly (butylene terephthalate), poly(ethylene 2,6-naphthalene dicarboxylate) (PEN), and poly (ethylene terephthalate).

Representative polyamides include crystalline or amorphous polyamides such as, but not limited to, nylon-6, nylon-6,6, nylon-6,9, nylon-6,10, aromatic nylon MXD6 (manufactured by Mitsubishi Gas Chemical America, Inc.), and mixtures thereof.

Representative polyacrylates include, but are not limited to, poly(methylmethacrylate) and polymethacrylate.

The particle can be a mixture of the aforementioned polymers. For example, the polymer can comprise about 70% to about 99% by weight acrylonitrile and about 30% to about 1% by weight styrene. Similarly, copolymers of vinyl chloride and vinylidene chloride with a vinyl chloride content of about 1 to about 30 mole percent and PET/PHB copolymers with a PHB content of about 60 to about 80 mole percent function effectively.

Methods for Applying the Compositions to the Device

Before applying the primer layer, the surface of the device or prosthesis should be clean and free from contaminants that may be introduced during manufacturing. However, the surface of the prosthesis requires no particular surface treatment to retain the applied coating. Metallic surfaces of stents can be, for example, cleaned by argon plasma process as is well known to one of ordinary skill in the art. Application of the composition can be by any conventional method, such as by spraying the composition onto the prosthesis or immersing the prosthesis in the composition. Operations such as wiping, centrifugation, blowing, or other web clearing acts can also be performed to achieve a more uniform coating. Briefly, wiping refers to physical removal of excess coating from the surface of the stent; centrifugation refers to rapid rotation of the stent about an axis of rotation; and blowing refers to application of air at a selected pressure to the deposited coating. The excess coating can also be vacuumed off the surface of the device. The addition of a wetting fluid leads to a consistent application of the composition, which also causes the coating to be uniformly deposited on the surface of the prosthesis.

With the use of the thermoplastic polymers, such as EVAL, polycaprolactone, poly(lactide-co-glycolide), poly(hydroxybutyrate), etc., the deposited primer composition can be exposed to a heat treatment at a temperature range greater than about the glass transition temperature (T_{g}) and less than about the melting temperature (Tₘ) of the selected polymer. Unexpected results have been discovered with treatment of the composition under this temperature range, specifically strong adhesion or bonding of the coating to the metallic surface of a stent. The device should be exposed to the heat treatment for any suitable duration of time, which would allow for the formation of the primer coating on the surface of the device and allows for the evaporation of the solvent or combination of solvent and wetting fluid. It is understood that essentially all of the solvent and the wetting fluid will be removed from the composition but traces or residues can remain blended with the polymer.

Table 3 lists the T_{g} and Tₘ for some of the polymers used in the embodiments of the present invention. T_{g} and Tₘ of polymers are attainable by one of ordinary skill in the art. The cited exemplary temperature and time for exposure is provided by way of illustration and it is not meant to be limiting.

TABLE 3

| | | | | Exemplary |
|---|---|---|---|---|
| | | | Exemplary | Duration of |
| | | | Temperature | Time For |
| Polymer | T_{g} (° C.) | Tₘ (° C.) | (° C.) | Heating |
| EVAL | 55 | 165 | 140 | 4 hours |
| Polycaprolactone | −60 | 60 | 50 | 2 hours |
| ethylene | 36 | 63 | 45 | 2 hours |
| vinylacetate | | | | |
| (e.g., 33% | | | | |
| vinylacetate content) | | | | |
| Polyvinyl alcohol | 75-85* | 200-220* | 165 | 2 hours |

*Exact temperature depends on the degree of hydrolysis which is also known as the amount of residual acetate.

With the use of one of the aforementioned thermoset polymers, the use of initiators may be required. By way of example, epoxy systems consisting of diglycidyl ether of bisphenol A resins can be cured with amine curatives, thermoset polyurethane prepolymers can be cured with polyols, polyamines, or water (moisture), and acrylated urethane can be cured with UV light. If baked, the temperature can be above the T_{g} of the selected polymer.

With the use of the inorganic polymers, such as silanes, titanates, and zirconates the composition containing the prepolymer or precursor is applied and the solvent is allowed to evaporate.

As the primer layer is being applied or after the primer coating has been formed, the surface of the primer can be modified in order to increase the surface area of the primer. For instance, in one embodiment, asperities, or areas of roughness, are created on the surface of the primer layer. A variety of methods can be used to create the asperities on the primer layer covering the outer surface of the prosthesis. In one method, a pressurized stream of grit material is directed upon the polymeric primer coating after the primer layer has been dried. Examples of such processes include bead blasting and sand blasting. Bead blasting refers to the use of pressurized gas to project beads of a relatively uniform diameter at an object at a high velocity. The beads may be made of materials such as, but not limited to, aluminum oxide, silicon oxide, or latex. In sand blasting, the grit projected does not have as uniform diameter as in bead blasting. Both bead blasting and sand blasting are techniques that are well known to those of ordinary skill in the art. The roughness achieved using a pressurized grit source can be controlled by the size of the grit, e.g., the diameter of the beads, the pressure used, the distance between the grit source and the primer surface and the length of time the grit is blasted at the primer surface. By way of example and not limitation, the grit can be beads having a diameter of between 10 μm and 50 μm. Pressures of 30 PSI (pounds per square inch) to 60 PSI can be used to project the beads from a distance of approximately 3 to 10 cm from the stent.

Laser etching can also be used to create asperities or pores on the primer coating after the primer layer has been dried. Laser lithographic methods are known to those of ordinary skill in the art. A laser is directed onto the primer coating for a predetermined period of time, which depends on the etch rate and the depth of etch desired. A patterned mask that has openings may be applied over the primer coating before the laser is utilized. The laser is then allowed to etch the primer through the openings of the mask. The use of patterned masks with laser etchings is known to those of ordinary skill in the art.

In addition, the manner in which the primer is deposited onto the outer surface of the stent can create the asperities. The primer may be added via physical deposition processes, for example, by sputtering. Process conditions in which a lower pressure and shorter deposition time than is typically used for thin film deposition are used to form the asperities in the primer coating.

In addition, the surface of the primer coating can be modified by using a method to form a porous matrix. A porous matrix for the primer coating can be provided, for example, by phase inversion precipitation of a portion of the polymer in the primer layer. By way of example, a polymer is mixed with two miscible solvents to form a solution. One of the solvents (solvent A) should be less volatile than the other solvent (solvent B). Additionally, the polymer should be less soluble in solvent A. The solution can then be applied to a portion of the surface of the implantable device. Next, when the solvents are allowed to evaporate, the polymer slowly precipitates as solvent B is essentially removed from the coating. As a result, after complete drying, the polymer matrix becomes porous. One of ordinary skill in the art will understand that the size of the pores can be controlled by the choice of polymers and solvents and the relative concentrations of the solutions. The depth of porous matrix into the primer region can be controlled by only using the phase inversion technique after a portion of the primer layer has been applied to the surface of the stent. Pores in the range of about 0.1 microns to about 1 micron in diameter may be suitable.

The porous matrix in the primer layer can also be formed by using porogens. For example, a porogen can be added to the primer composition as monodispersed particles to create a porogen suspension. The porogen particles can comprise from about 5% to about 50% by weight of the total weight of the primer composition. The size of the porogen particles can be about 0.1 microns to 2 microns. The composition can then be applied to the device and after the layer is formed, the porogens can be dissolved away with an appropriate solvent, leaving cavities or pores in the primer layer to form the porous matrix. Representative examples of porogen particles include sodium chloride and glycine spheres. Usefully, the appropriate solvent is a compatible solvent for the porogen, but does not substantially dissolve the polymer used to form the primer layer. Water is a representative example of an appropriate solvent if sodium chloride or glycine spheres are used as the porogen.

The porous matrix can also be formed by using a sintering process. Sintering is a process of fabrication where particles are bonded together by partially melting some of the particles. For example, a polymeric powder or particles can be applied to the surface of the device and then pressed together. The particles can usefully be about 1 micron to about 10 microns. Then, the polymeric particles can be heated to temperatures slightly below or about the melting point of the polymer. Without entirely melting all of the particles, the particles bond to each other at their respective surfaces. Space remains between the lattice of the particles to form porous cavities.

Subsequent to the application of the primer layer, the composition containing the active ingredient (i.e., reservoir layer) can be applied to a designated portion of the primer coating. Masking techniques can be implemented for applying compositions containing different active ingredients to selected portions of the primer layer. Accordingly, stents having various cocktail formulations or combinations of a variety of active ingredients can be manufactured. The solvent(s) or the combination of the solvent(s) and the wetting fluid is removed from the composition by allowing the solvent(s) or combination of the solvent(s) and the wetting fluid to evaporate. The evaporation can be induced by heating the device at a predetermined temperature for a predetermined period of time. For example, the device can be heated at a temperature of about 60° C. for about 12 hours to about 24 hours. The heating can be conducted in an anhydrous atmosphere and at ambient pressure and should not exceed the temperature which would adversely affect the active ingredient. The heating can, alternatively, be conducted under a vacuum condition. It is understood that essentially all of the solvent and the wetting fluid will be removed from the composition containing the active ingredient but traces or residues can remain blended with the polymer.

A diffusion barrier layer can also be applied on a designated portion of the active ingredient-containing coating subsequent to the evaporation of the solvent(s) or solvent(s)/wetting fluid and the drying of the polymer for the active ingredient-containing coating. The diffusion barrier layer can also be applied by spraying the composition onto the device or immersing the device in the composition. The above-described processes can be similarly repeated for the formation of the barrier region.

Method of Use

In accordance with the above-described method, the active ingredient can be applied to a medical device, e.g., a stent, retained on the stent during delivery and expansion of the stent, and released at a desired control rate and for a predetermined duration of time at the site of implantation. A stent having the above-described coating layers is useful for a variety of medical procedures, including, by way of example, treatment of obstructions caused by tumors in bile ducts, esophagus, trachea/bronchi and other biological passageways. A stent having the above-described coating layers is particularly useful for treating occluded regions of blood vessels caused abnormal or inappropriate migration and proliferation of smooth muscle cells, thrombosis, and restenosis. Stents may be placed in a wide array of blood vessels, both arteries and veins. Representative examples of sites include the iliac, renal, and coronary arteries. The application of the present invention should not, however, be limited to stents such that the embodiments of the coating can be used with a variety of medical substrates.

While particular embodiments of the present invention have been shown and described, it will be obvious to those skilled in the art that changes and modifications can be made without departing from the embodiments of this invention in its broader aspects and, therefore, the appended claims are to encompass within their scope all such changes and modifications as fall within the true spirit and scope of the embodiments of this invention. Additionally, various embodiments have been described above. For convenience's sake, combinations of aspects (such as monomer type or gas flow rate) composing invention embodiments have been listed in such a way that one of ordinary skill in the art may read them exclusive of each other when they are not necessarily intended to be exclusive. But a recitation of an aspect for one embodiment is meant to disclose its use in all embodiments in which that aspect can be incorporated without undue experimentation. In like manner, a recitation of an aspect as composing part of an embodiment is a tacit recognition that a supplementary embodiment exists in which that aspect specifically excludes that aspect.

Moreover, some embodiments recite ranges. When this is done, it is meant to disclose the ranges as a range, and to disclose each and every point within the range, including end points. For those embodiments that disclose a specific value or condition for an aspect, supplementary embodiments exist that are otherwise identical, but that specifically include the value or the conditions for the aspect.

1. An implantable device comprising a coating for delivery of an active ingredient, wherein the coating includes:
a primer region comprising a polymer on at least a portion of a surface of an implantable device, wherein the primer region has a thickness X;
a reservoir region comprising a polymer and an active ingredient on at least a selected portion of the primer region, wherein the reservoir region has a thickness Y; and
wherein the thickness X is measured from the outer surface of the primer region to the surface of the implantable device prior to the migration of the active ingredient from the reservoir region to the primer region, and
wherein
(a) the thickness X is about 0.1 microns to about 10 microns and the thickness Y is about 0.5 micron to about 10 microns,
(b) X/Y is selected from the following ranges: 0.01 to 0.2; 0.05 to 0.2; 0.01 to 20; 0.05 to 20; 0.01 to 1; 0.05 to 1; or 0.01 to 0.05, or
both (a) and (b).

2. The implantable device of claim 1 the thickness X is about 0.1 microns to about 10 microns and the thickness Y is about 0.5 micron to about 10 microns

3. The implantable device of claim 1 wherein X/Y is equal to or greater than 0.01 and equal to or less than 20.

4. The implantable device of claim 1 wherein X/Y is equal to or greater than 0.01 and equal to or less than 0.2.

5. The implantable device of claim 1 wherein X/Y is equal to or greater than 0.05 and equal to or less than 20.

6. The implantable device of claim 1 wherein X/Y is equal to or greater than 0.05 and equal to or less than 0.2.

7. The implantable device of claim 1 wherein the primer region or the reservoir region comprises an ethylene vinyl alcohol copolymer or poly(butyl methacrylate).

8. The implantable device of claim 1 further comprising forming a barrier region on at least a selected portion of the reservoir region to reduce the rate at which the active ingredient is released from the reservoir region after insertion of the device into a body of a patient.

9. The implantable device of claim 1 wherein the device is a stent.

10. The implantable device of claim 3 wherein the device is a stent.

11. The implantable device of claim 5 wherein the device is a stent.

12. The implantable device of claim 7 wherein the device is a stent.

13. A method of forming a coating for an implantable device, comprising:
forming a primer region comprising a polymer on at least a portion of a surface of an implantable device wherein the primer region has a thickness of X;
forming a reservoir region comprising a polymer and an active ingredient on at least a selected portion of the primer region wherein the reservoir region has a thickness of Y; and
wherein
(a) the thickness X is about 0.1 microns to about 10 microns and the thickness Y is about 0.5 micron to about 10 microns,
(b) X/Y is selected from the following ranges: 0.01 to 0.2; 0.05 to 0.2; 0.01 to 20; 0.05 to 20; 0.01 to 1; 0.05 to 1; or 0.01 to 0.05, or
both (a) and (b).

14. The method of claim 13 wherein the thickness X is about 0.1 microns to about 10 microns and the thickness Y is about 0.5 micron to about 10 microns.

15. The method of claim 13 wherein X/Y is equal to or greater than 0.01 and equal to or less than 20.

16. The method of claim 13 wherein X/Y is equal to or greater than 0.01 and equal to or less than 0.2.

17. The method of claim 13 wherein X/Y is equal to or greater than 0.05 and equal to or less than 20.

18. The method of claim 13 wherein X/Y is equal to or greater than 0.05 and equal to or less than 0.2.

19. The method of claim 13 further comprising forming a barrier region on at least a selected portion of the reservoir region to reduce the rate at which the active ingredient is released from the reservoir region after insertion of the device into a body of a patient.

20. The method of claim 13 wherein the primer region or the reservoir region comprises an ethylene vinyl alcohol copolymer or poly(butyl methacrylate).

21. An implantable device comprising a coating for delivery of an active ingredient, wherein the coating includes:
a primer region comprising a polymer on at least a portion of a surface of an implantable device, wherein the primer region has a mass X;
a reservoir region comprising a polymer and an active ingredient on at least a selected portion of the primer region, wherein the reservoir region has a mass Y; and
wherein
X/Y is selected from the following ranges: 0.01 to 0.2; 0.05 to 0.2; 0.01 to 20; 0.05 to 20; 0.01 to 1; 0.05 to 1; or 0.01 to 0.05.

22. The implantable device of claim 21 wherein X/Y is equal to or greater than 0.01 and equal to or less than 20.

23. The implantable device of claim 21 wherein X/Y is equal to or greater than 0.01 and equal to or less than 0.2.

24. The implantable device of claim 21 wherein X/Y is equal to or greater than 0.05 and equal to or less than 20.

25. The implantable device of claim 21 wherein X/Y is equal to or greater than 0.05 and equal to or less than 0.2.

26. The implantable device of claim 21 wherein the primer region or the reservoir region comprises an ethylene vinyl alcohol copolymer or poly(butyl methacrylate).

27. The implantable device of claim 21 further comprising forming a barrier region on at least a selected portion of the reservoir region to reduce the rate at which the active ingredient is released from the reservoir region after insertion of the device into a body of a patient.

28. The implantable device of claim 21 wherein the device is a stent.

29. The implantable device of claim 22 wherein the device is a stent.

30. The implantable device of claim 26 wherein the device is a stent.

31. The implantable device of claim 24 wherein the device is a stent.

32. A method of forming a coating for an implantable device, comprising:
forming a primer region comprising a polymer on at least a portion of a surface of an implantable device wherein the primer region has a mass X;
forming a reservoir region comprising a polymer and an active ingredient on at least a selected portion of the primer region wherein the reservoir region has a mass Y; and
wherein X/Y is selected from the following ranges: 0.01 to 0.2; 0.05 to 0.2; 0.01 to 20; 0.05 to 20; 0.01 to 1; 0.05 to 1; or 0.01 to 0.05.

33. The method of claim 32 wherein X/Y is equal to or greater than 0.01 and equal to or less than 20.

34. The method of claim 32 wherein X/Y is equal to or greater than 0.01 and equal to or less than 0.2.

35. The method of claim 32 wherein X/Y is equal to or greater than 0.05 and equal to or less than 20.

36. The method of claim 32 wherein X/Y is equal to or greater than 0.05 and equal to or less than 0.2.

37. The method of claim 32 further comprising forming a barrier region on at least a selected portion of the reservoir region to reduce the rate at which the active ingredient is released from the reservoir region after insertion of the device into a body of a patient.

38. The method of claim 32 wherein the primer region or the reservoir region comprises an ethylene vinyl alcohol copolymer or poly(butyl methacrylate).

39. An implantable device comprising a coating for delivery of an active ingredient, wherein the coating includes:
a primer region comprising a polymer on at least a portion of a surface of an implantable device, wherein the primer region has a mass X;
a reservoir region comprising a polymer and an active ingredient on at least a selected portion of the primer region, wherein the reservoir region has a mass Y exclusive of the active ingredient; and
wherein
X/Y is selected from the following ranges: 0.01 to 0.2; 0.05 to 0.2; 0.01 to 20; 0.05 to 20; 0.01 to 1; 0.05 to 1; or 0.01 to 0.05.

40. The implantable device of claim 39 wherein X/Y is equal to or greater than 0.01 and equal to or less than 20.

41. The implantable device of claim 39 wherein X/Y is equal to or greater than 0.01 and equal to or less than 0.2.

42. The implantable device of claim 39 wherein X/Y is equal to or greater than 0.05 and equal to or less than 20.

43. The implantable device of claim 39 wherein X/Y is equal to or greater than 0.05 and equal to or less than 0.2.

44. The implantable device of claim 39 wherein the primer region or the reservoir region comprises an ethylene vinyl alcohol copolymer or poly(butyl methacrylate).

45. The implantable device of claim 39 further comprising forming a barrier region on at least a selected portion of the reservoir region to reduce the rate at which the active ingredient is released from the reservoir region after insertion of the device into a body of a patient.

46. The implantable device of claim 39 wherein the device is a stent.

47. The implantable device of claim 41 wherein the device is a stent.

48. The implantable device of claim 42 wherein the device is a stent.

49. The implantable device of claim 44 wherein the device is a stent.

50. A method of forming a coating for an implantable device, comprising:
forming a primer region comprising a polymer on at least a portion of a surface of an implantable device wherein the primer region has a mass X;
forming a reservoir region comprising a polymer and an active ingredient on at least a selected portion of the primer region wherein the reservoir region has a mass Y exclusive of the active ingredient; and
wherein
(i) X/Y is selected from the following ranges: 0.01 to 0.2; 0.05 to 0.2; 0.01 to 20; 0.05 to 20; 0.01 to 1; 0.05 to 1; or 0.01 to 0.05.

51. The method of claim 50 wherein X/Y is equal to or greater than 0.01 and equal to or less than 20.

52. The method of claim 50 wherein X/Y is equal to or greater than 0.01 and equal to or less than 0.2.

53. The method of claim 50 wherein X/Y is equal to or greater than 0.05 and equal to or less than 20.

54. The method of claim 50 wherein X/Y is equal to or greater than 0.05 and equal to or less than 0.2.

55. The method of claim 50 further comprising forming a barrier region on at least a selected portion of the reservoir region to reduce the rate at which the active ingredient is released from the reservoir region after insertion of the device into a body of a patient.

56. The method of claim 50 wherein the primer region or the reservoir region comprises an ethylene vinyl alcohol copolymer or poly(butyl methacrylate).
